SEQUENCE LISTING (6 sequences)

SEQ ID NO: 1 - protein, 94 aa, Homo sapiens
  ref <301>: AUTHORS: Ulvsback, M., Lindstrom, C., Weiber, H., Abrahamson, P.A., Lilja, H., and Lundwall, A“
  ref <302>: TITLE: Molecular cloning of a small prostate protein, known as beta-microsemenoprotein, PSP94 or beta-inhibin, and demonstration of transcripts in non-genital tissues.
  ref <303>: JOURNAL: Biochem. Biophys. Res Commun.
  ref <304>: VOLUME: 164
  ref <305>: ISSUE: 3
  ref <306>: PAGES: 1310-1315
  ref <307>: DATE: 1989
  ref <308>: DATABASE ACCESSION NUMBER: GI 131436
  ref <309>: DATABASE ENTRY DATE: 1988-08-01
SCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 2 - protein, 102 aa, Artificial Sequence
  recombinant human PSP94 (rHuPSP94) produced from yeast
EAEAYVEFSCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 3 - protein, 10 aa, Artificial Sequence
  decapeptide
YTCSVSEPGI

SEQ ID NO: 4 - protein, 15 aa, Artificial Sequence
  Polypeptide 7-21
NEGVPGDSTRKCMDL

SEQ ID NO: 5 - protein, 15 aa, Artificial Sequence
  PCK3145 (polypeptide 31-45)
EWQTDNCETCTCYET

SEQ ID NO: 6 - protein, 19 aa, Artificial Sequence
  Polypeptide 76-94
IVVEKKDPKKTCSVSEWII

(19) United States
(12) Patent Application Publication
(10) Pub. No.: US 2003/0119744 A1
(43) Pub. Date: Jun. 26, 2003

(54) PSP-94: use for treatment of hypercalcemia and bone metastasis

(76) Inventors: Shafaat Ahmed Rabbani, Westmount (CA); Nicholas Shukeir, Montreal (CA); Chandra J. Panchal, London (CA); Christopher Newman, Beaconsfield (CA)

Correspondence Address: BROUILLETTE KOSIE PRINCE, 25th Floor, 1100 Rene-Levesque Boulevard West, Montreal, QC, H3B 5C9, CA

(21) Appl. No.: 10/291,360

(22) Filed: Nov. 8, 2002

(30) Foreign Application Priority Data
Nov. 8, 2001 (CA) 2,361,736

(51) Int. Cl. A61K 38/17

(52) U.S. Cl. 514/12

(57) ABSTRACT

The present invention discloses the use of PSP-94 for treating a patient suffering from hypercalcemia of malignancy. Prostate secretory protein of 94 amino acids (PSP-94) is known to serve as prognostic marker in hormone-dependent prostate. In the current study the effect of PSP-94 on prostate cancer growth, metastases to the skeleton and hypercalcemia of malignancy was examined. A marked dose-dependent decrease in primary tumor volume was seen in experimental animals receiving PSP-94. Furthermore, while control animals routinely developed hypercalcemia due to PTHrP production, treatment with PSP-94 led to a near normalization of plasma calcium and a marked reduction in PTHrP production as determined by radioimmunoassay and immunohistochemistry. Results obtained demonstrate the ability of PSP-94 to be an effective treatment modality for prostate cancer. Furthermore, decrease in plasma PTHrP and calcium levels can serve as useful biochemical markers for monitoring the efficacy of this novel anti-tumor agent.

### BACKGROUND OF THE INVENTION

The prostate gland, which is found exclusively in male mammals, produces several components of semen and blood and several regulatory peptides. The prostate gland comprises stroma and epithelium cells, the latter group consisting of columnar secretory cells and basal nonsecretory cells. A proliferation of these basal cells as well as stroma cells gives rise to benign prostatic hyperplasia (BPH), which is one common prostate disease. Another common prostate disease is prostatic adenocarcinoma (CaP), which is the most common of the fatal pathophysiological prostate cancers, and involves a malignant transformation of epithelial cells in the peripheral region of the prostate gland. Prostatic adenocarcinoma and benign prostatic hyperplasia are two common prostate diseases, which have a high rate of incidence in the aging human male population. Approximately one out of every four males above the age of 55 suffers from a prostate disease of some form or another. Prostate cancer is the second most common cause of cancer related death in elderly men, with approximately 96,000 cases diagnosed and about 26,000 deaths reported annually in the United States.

A distinct feature of prostate cancer is its ability to cause osteoblastic skeletal metastases which contributes to the high rate of morbidity and mortality associated with this hormone dependent malignancy. Additionally, a significant number of patients with prostate cancer exhibit an increase in their plasma calcium levels due to the production of PTHrP by tumor cells. Hypercalcemia has been recognized as a complication of malignancy since 1920 and occurs in at least 15-20% of patients harbouring a variety of cancers including prostate cancer. Although no single agent has been shown to be uniquely responsible for the hypercalcemia of malignancy (HM), increased production of parathyroid hormone related peptide (PTHrP) by tumor cells has led to its establishment as the major pathogenetic factor responsible for HM. This is of particular significance in prostate and breast cancer which are often associated with skeletal metastasis where osteolytic effects of PTHrP results in increased bone resorption and hypercalcemia.

Clinical prostate cancer can be treated successfully at its early stage when the cancer is well confined within the prostate gland. However, increased production of many factors including growth factors, sex steroids, angiogenic factors and proteases such as urokinase (uPA) and matrix metalloproteinases (MMPs) by tumor cells and their surrounding stroma is associated with high mortality. Despite recent advances in the therapeutic modalities for organ confined prostate cancer including surgery and radiotherapy, limited success has been obtained in treating hormone-independent metastatic prostate cancer.

Prostate specific antigen (PSA) and prostate secretory protein of 94 amino acids (herein referred to PSP-94 or PSP) are known to serve as prognostic markers for disease progression. Like PSA, PSP-94 levels in serum, urine, and prostate tissue of patients with prostate cancer are inversely related to tumor grade. In previous work, described in U.S. Pat. No. 5,428,011 (the entire content of which is incorporated herein by reference), pharmaceutical preparations (i.e., compositions) of native human seminal plasma PSP-94 were provided for inhibiting in-vitro and in-vivo cancerous prostate, gastrointestinal and breast tumors. In additional work disclosed in Canadian patent application No: 2,359,650 (the entire content of wich is incorporated herein by reference), the ability of PSP-94 fragments, such as PCK3145, to be used in the inhibition of tumor growth and more particularly, in the inhibition of prostate cancer tumor growth was illustrated.

In the present study, the effect of PSP-94 on prostate cancer tumor growth and especially bone metastases was evaluated. For these studies syngeneic in vivo model of rat prostate cancer using the rat prostate cancer cell line Dunning R3227 Mat Ly Lu transfected with the full length cDNA encoding rat PTHrP was used (Rabbani, S. A. et al., Int. J. Cancer, 80: 257-264, 1999). Following sub-cutaneous (S.C.) or intracardiac (I.C.) inoculation of Mat Ly Lu-PTHrP cells, the ability of different doses of PSP-94 or PCK3145 to reduce tumor growth, metastases, tumoral PTHrP production, plasma calcium and plasma PTHrP was evaluated. Amino acid sequence homology between human and rat PSP and similarity in their tertiary structures as determined by highly conserved cysteine residues has allowed the use of human PSP-94 for these studies (Fernlund, P. et al., Arch. Biochem. Biophys. 334:73-82, 1996). Due to the high levels of PTHrP production these animals routinely develop hypercalcemia, a common complication in many patients suffering from prostate cancer (Iwamura, M., et al., Urology, 43: 675-679, 1994; Iwamura, M. et al., Hum. Pathol. 26: 797-801, 1995). Use of this homologous model for prostate cancer allows for full interaction between the host environment and growth factors (EGF, TGF-β) (Helawell, G. O. et al., BJU Int. 89:230-240,2002) and proteases (uPA, MMPs) (Rabbani, S. A., et al., Int. J. Cancer, 87: 276-282, 2000, Rabbani, S. A., et al., In vivo, 12:135-142, 1998) secreted by tumor cells. These prostate cancer cells are hormone-independent allowing for the evaluation of the effect of PSP-94 on late stage prostate cancer.

### SUMMARY OF THE INVENTION

The invention disclosed herein provides pharmaceutical compositions and method for treating patients with hypercalcemia of malignancy and skeletal metastasis. PSP-94 (native PSP-94 (nPSP-94) (SEQ ID NO. 1)) and rHuPSP94 (recombinant human PSP-94 (SEQ ID No. 2)) as well as derivatives (fragments) such as for example the decapeptide as set forth in SEQ ID NO: 3, the polypeptide as set forth in SEQ ID NO: 4 (polypeptide 7-21), the polypeptide as set forth in SEQ ID NO: 5 (PCK3145), the polypeptide as set forth in SEQ ID NO: 6 (polypeptide 76-94), and polypeptide analogs are used herein to treat conditions related to hypercalcemia and skeletal metastasis. Calcium may also be used herein as a surrogate marker of the efficacy of PSP-94 tumor treatment.

In a first aspect, the present invention relates the use of PSP-94 and analog thereof for treating a patient (with a malignancy) suffering from hypercalcemia of malignancy (i.e., for treating hypercalcemia of malignancy). More particularly, the present invention relates to the use of a polypeptide selected from the group consisting of PSP-94, PCK3145, the polypeptide 7-21, the decapeptide, the polypeptide 76-94, and analog thereof to reduce (treat a patient with) hypercalcemia (related to) of malignancy. This aspect of the invention also encompass methods for treating a patient suffering from hypercalcemia of malignancy which comprise administering to the patient a pharmaceutical composition comprising PSP-94, the polypeptides mentioned herein and analogs thereof. This aspect of the invention also encompass the use of PSP-94 for reducing (lowering) calcium levels in a patient suffering from hypercalcemia of malignancy.

In accordance with the present invention, the malignancy may be an hormone-independent malignancy. It is to be understood herein that hypercalcemia of malignancy may arise from various source including prostate cancer, breast cancer, lung carcinoma, hepatocellular carcinoma, etc. Therefore, treatment of hypercalcemia of malignancy with PSP-94, the polypeptide described herein and analogs thereof may be necessary in patient with prostate cancer, breast cancer, lung carcinoma, hepatovellular carcinoma, etc. Treatment of hypercalcemia of malignancy is not restricted to any type of malignancy.

In another aspect, the present invention relates to the use of PSP-94 to prevent occurrence of hypercalcemia of malignancy and to control the induction (onset) of hypercalcemia in a patient.

In yet another aspect, the present invention relates to the use of PSP-94 to prevent (control) PTHrP increase in a patient.

In an additional aspect, the present invention relates to the use of a polypeptide selected from the group consisting of PSP-94, PCK3145, the polypeptide 7-21, the decapeptide, the polypeptide 76-94, and analog thereof to reduce (for reducing/lowering) the level (biosynthesis, expression, transcription, translation, production, secretion) or activity of PTHrP in a patient in need thereof

In a further aspect, the present invention relates to the use of a polypeptide selected from the group consisting of PSP-94, PCK3145, the polypeptide 7-21, the decapeptide, the polypeptide 76-94, and analog thereof to reduce the production of agents responsible for the development of (an hypercalcemic condition) hypercalcemia including PTHrP.

In a further aspect, the present invention relates to the use of PSP-94 to reduce (delay) the development of skeletal metastasis. More particularly, the present invention relates to the use of a polypeptide selected from the group consisting of PSP-94, PCK3145, the polypeptide 7-21, the decapeptide, the polypeptide 76-94, and analog thereof to block (reduce, impair, delay) the development (progression) of skeletal metastasis. This aspect of the invention also encompass methods for treating a patient with skeletal metastasis comprising administering to the patient a pharmaceutical composition comprising PSP-94, the polypeptides described herein and analogs thereof.

In an additional aspect, the present invention relates to the use of a polypeptide selected from the group consisting of PSP-94, PCK3145, the polypeptide 7-21, the decapeptide, the polypeptide 76-94, and analog thereof to control the level of molecules involved in calcium production, wherein said molecules are selected from the group consisting of vitamine B, calcitonine and biological equivalents thereof.

In yet an additional aspect, the present invention relates to the use of a polypeptide selected from the group consisting of PSP-94, PCK3145, the polypeptide 7-21, the decapeptide, the polypeptide 76-94, and analog thereof conjugated with bisphosphonates, RGD peptides (Arginine-Glycine-Aspartic acid peptides), osteoblast, and osteoclast specific proteins to improve their bioavailibility to the skeleton.

In another aspect, the present invention relates to a pharmaceutical composition comprising;

a) a polypeptide selected from the group consisting of PSP-94, PCK3145, the polypeptide 7-21, the decapeptide, the polypeptide 76-94, and analog thereof; and

b) a pharmaceutically acceptable carrier,

for the treatment of hypercalcemia of malignancy or for the treatment of skeletal metastasis.

In a further aspect, the present invention relates to the use of PSP-94 in the manufacture of a pharmaceutical composition for the treatment of hypercalcemia of malignancy. More particularly, the present invention relates to the use of a polypeptide selected from the group consisting of PSP-94, PCK3145, the polypeptide 7-21, the decapeptide, the polypeptide 76-94, and analog thereof for the manufacture of a pharmaceutical composition for the treatment of hypercalcemia of malignancy.

In yet a further aspect, the present invention relates to the use of PSP-94 in the manufacture of a pharmaceutical composition for treatment of skeletal metastasis. More particularly, the present invention relates to the use of a polypeptide selected from the group consisting of PSP-94, PCK3145, the polypeptide 7-21, the decapeptide, the polypeptide 76-94, and analog thereof for the manufacture of a pharmaceutical composition for the treatment of skeletal metastasis.

In an additional aspect, the present invention relates to a method of treating a patient with a condition related to hypercalcemia of malignancy comprising administering to the patient a pharmaceutical composition comprising a polypeptide selected from the group consisting of PSP-94, PCK3145, the polypeptide 7-21, the decapeptide, the polypeptide 76-94, and analog thereof and a pharmaceutically acceptable carrier.

In yet an additional aspect, the present invention relates to a method of treating a patient with skeletal metastasis comprising administering to the patient a pharmaceutical composition comprising a polypeptide selected from the group consisting of PSP-94, PCK3145, the polypeptide 7-21, the decapeptide, the polypeptide 76-94, and analog thereof and a pharmaceutically acceptable carrier.

In a further aspect, the present invention relates to the use of polypeptide selected from the group consisting of PSP-94, PCK3145, the polypeptide 7-21, the decapeptide, the polypeptide 76-94, and analog thereof in combination with hormone therapy, chemotherapy or radiation therapy.

In accordance with the present invention, PSP-94 may be selected from the group consisting of native PSP-94 (nPSP-94) and rHuPSP94.

In accordance with the present invention, the polypeptide may be used with an antibody, an hormone or an anticancer drug, including for example, (without being restricted to) mitomycin, idarubicin, cisplatin, 5-fluoro-uracil, methotrexate, adriamycin, daunomycin, taxol (i.e., paclitaxel), and taxol derivative (e.g.,docetaxel, taxane).

In another aspect, the present invention relates to a method for evaluating, the efficacy of PSP-94 tumor treatment in (of) a patient having a tumor, said method comprising measuring plasma calcium levels of said patient.

In a further aspect the present application relates to a method for evaluating, in a patient the efficacy of PSP-94 treatment of hypercalcemia of malignancy, said method comprising measuring plasma calcium levels (in) of said patient.

In yet a further aspect, the present invention relates to a method for evaluating, in a patient, the efficacy of PSP-94 treatment, said method comprising;

a) measuring plasma calcium from a patient with a tumor or with hypercalcemia of malignancy before the patient's treatment with PSP-94,

b) measuring plasma calcium from a patient with a tumor or with hypercalcemia of malignancy after the patient's treatment with PSP-94; and

c) comparing values obtained in step a) with values obtained in step b).

In another aspect, the present invention relates to a method for evaluating, the efficacy of PSP-94 tumor treatment in (of) a patient having a tumor, said method comprising measuring plasma PTHrP levels of said patient.

In a further aspect the present application relates to a method for evaluating, in a patient, the efficacy of PSP-94 treatment of hypercalcemia of malignancy, said method comprising measuring plasma PTHrP levels (in) of said patient.

In yet a further aspect, the present invention relates to a method for evaluating, in a patient, the efficacy of PSP-94 treatment, said method comprising;

a) measuring plasma PTHrP (levels) from a patient with a tumor or with hypercalcemia of malignancy before his (the patient's) treatment with PSP-94,

b) measuring plasma PTHrP (levels) from a patient with a tumor or with hypercalcemia of malignancy after his (the patient's) treatment with PSP-94; and

c) comparing values obtained in step a) with values obtained in step b).

As used herein, “polypeptides” refers to any peptide or protein comprising two or more amino acids joined to each other by peptide bonds or modified peptide bonds (i.e., peptide isosteres). “Polypeptide” refers to both short chains, commonly referred as peptides, oligopeptides or oligomers, and to longer chains generally referred to as proteins. As described above, polypeptides may contain amino acids other than the 20 gene-encoded amino acids.

As used herein, the term “tumor” relates to solid or non-solid tumors, metastasic or non-metastasic tumors, tumors of different tissue origin including, but not limited to, tumors originating in the liver, lung, brain, lymph node, bone marrow, adrenal gland, breast, colon, pancreas, prostate, stomach, or reproductive tract (cervix, ovaries, endometrium etc.). The term “tumor” as used herein, refers also to all neoplastic cell growth and proliferation, whether malignant or benign, and all pre-cancerous and cancerous cells and tissues.

As used herein, “pharmaceutical composition” means therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). Solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally, etc.

Further, as used herein “pharmaceutically acceptable carrier” or “pharmaceutical carrier” are known in the art and include, but are not limited to, 0.01-0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's orfixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Mutant (variant, analog, derivative) polypeptides encompassed by the present invention includes mutant that will possess one or more mutations, which are deletions (e.g., truncations), insertions (e.g., additions), or substitutions of amino acid residues. Mutants can be either naturally occurring (that is to say, purified or isolated from a natural source) or synthetic (for example, by performing site-directed mutagenesis on the encoding DNA or made by other synthetic methods such as chemical synthesis). It is thus apparent that the polypeptides of the invention can be either naturally occurring or recombinant (that is to say prepared from the recombinant DNA techniques). Mutant polypeptide derived from PSP-94 (native PSP-94 (nPSP-94); SEQ ID NO.: 1 or rHuPSP94 (recombinant human PSP-94): SEQ ID NO.: 2) as well as derived from the polypeptide described herein (PCK3145 (SEQ ID NO.: 5), decapeptide (SEQ ID NO.: 3), polypeptide 7-21 (SEQ ID NO. 4), polypeptide 76-94 (SEQ ID NO. 6)) having the biological activity described herein (effect on hypercalcemia and bone metastasis) are included in the present application.

As may be appreciated, a number of modifications may be made to the polypeptides and fragments of the present invention without deleteriously affecting the biological activity of the polypeptides or fragments. Polypeptides of the present invention comprises for example, those containing amino acid sequences modified either by natural processes, such as posttranslational processing, or by chemical modification techniques which are known in the art. Modifications may occur anywhere in a polypeptide including the polypeptide backbone, the amino acid side-chains and the amino or carboxy termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslational natural processes or may be made by synthetic methods. Modifications comprise for example, without limitation, acetylation, acylation, addition of acetomidomethyl (Acm) group, ADP-ribosylation, amidation, covalent attachment to fiavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular proterties, 2nd Ed., T. E. Creighton, W. H. Freeman and Company, New-York, 1993).

Other type of polypeptide modification may comprise, for example, amino acid insertion (i.e., addition), deletion and substitution (i.e., replacement), either conservative or non-conservative (e.g., D-amino acids, desamino acids) in the polypeptide sequence where such changes do not substantially alter the overall biological activity of the polypeptide. Polypeptides of the present invention comprise for example, biologically active mutants, variants, fragments, chimeras, and analogs; fragments encompass amino acid sequences having truncations of one or more amino acids, wherein the truncation may originate from the amino terminus (N-terminus), carboxy terminus (C-terminus), or from the interior of the protein. Analogs of the invention involve an insertion or a substitution of one or more amino acids. Variants, mutants, fragments, chimeras and analogs may have the biological property of polypeptides of the present invention which is to inhibit growth of prostatic adenocarcinoma, stomach cancer, breast cancer, endometrial, ovarian or other cancers of epithelial secretion, or benign prostate hyperplasia (BPH).

Example of substitutions may be those, which are conservative (i.e., wherein a residue is replaced by another of the same general type). As is understood, naturally occurring amino acids may be sub-classified as acidic, basic, neutral and polar, or neutral and non-polar. Furthermore, three of the encoded amino acids are aromatic. It may be of use that encoded polypeptides differing from the determined polypeptide of the present invention contain substituted codons for amino acids, which are from the same group as that of the amino acid be replaced. Thus, in some cases, the basic amino acids Lys, Arg and His may be interchangeable; the acidic amino acids Asp and Glu may be interchangeable; the neutral polar amino acids Ser, Thr, Cys, Gln, and Asn may be interchangeable; the non-polar aliphatic amino acids Gly, Ala, Val, lie, and Leu are interchangeable but because of size Gly and Ala are more closely related and Val, Ile and Leu are more closely related to each other, and the aromatic amino acids Phe, Trp and Tyr may be interchangeable.

It should be further noted that if the polypeptides are made synthetically, substitutions by amino acids, which are not naturally encoded by DNA may also be made. For example, alternative residues include the omega amino acids of the formula NH₂(CH₂)ₙCOOH wherein n is 2-6. These are neutral nonpolar amino acids, as are sarcosine, t-butyl alanine, t-butyl glycine, N-methyl isoleucine, and norleucine. Phenylglycine may substitute for Trp, Tyr or Phe; citrulline and methionine sulfoxide are neutral nonpolar, cysteic acid is acidic, and ornithine is basic. Proline may be substituted with hydroxyproline and retain the conformation conferring properties.

It is known in the art that mutants or variants may be generated by substitutional mutagenesis and retain the biological activity of the polypeptides of the present invention. These variants have at least one amino acid residue in the protein molecule removed and a different residue inserted in its place. For example, one site of interest for substitutional mutagenesis may include but are not restricted to sites identified as the active site(s), or immunological site(s). Other sites of interest may be those, for example, in which particular residues obtained from various species are identical. These positions may be important for biological activity. Examples of substitutions identified as “conservative substitutions” are shown in table 1. If such substitutions result in a change not desired, then other type of substitutions, denominated “exemplary substitutions” in table 1, or as further described herein in reference to amino acid classes, are introduced and the products screened.

In some cases it may be of interest to modify the biological activity of a polypeptide by amino acid substitution, insertion, or deletion. For example, modification of a polypeptide may result in an increase in the polypeptide's biological activity, may modulate its toxicity, may result in changes in bioavailability or in stability, or may modulate its immunological activity or immunological identity. Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:

(1) hydrophobic: norleucine, methionine (Met), Alanine (Ala), Valine (Val), Leucine (Leu), Isoleucine (Ile)

(2) neutral hydrophilic: Cysteine (Cys), Serine (Ser), Threonine (Thr)

(3) acidic: Aspartic acid (Asp), Glutamic acid (Glu)

(4) basic: Asparagine (Asn), Glutamine (Gln), Histidine (His), Lysine (Lys), Arginine (Arg)

(5) residues that influence chain orientation: Glycine (Gly), Proline (Pro); and

(6) aromatic: Tryptophan (Trp), Tyrosine (Tyr), Phenylalanine (Phe)

Non-conservative substitutions will entail exchanging a member of one of these classes for another.

TABLE 1
Preferred amino acid substitution

| Original residue | Exemplary substitution | Conservative substitution |
|---|---|---|
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, | Leu |
| | norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, | Leu |
| | norleucine | |

| | | | | | | | | | | | | | | | |
|---|---|---|---|---|---|---|---|---|---|---|---|---|---|---|---|
| Position | 1 | | | | 5 | | | | | 10 | | | | | 15 |
| PCK3145 | E | W | Q | T | D | N | C | E | T | C | T | C | Y | E | T |
| | X₁ | W | Q | X₂ | D | X₁ | C | X₁ | X₂ | C | X₂ | C | X₃ | X₁ | X₂ |

For example, X₁ could be glutamic acid (i.e., glutamate) (Glu), aspartic acid (aspartate) (Asp), or asparagine (Asn), X₂ could be threonine (Thr) or serine (Ser) and X₃ could be tyrosine (Tyr) or phenylalanine (Phe).

Polypeptides that are polypeptide analogs of PSP-94 (nPSP-94 (SEQ ID NO.: 1) or rHuPSP94 (SEQ ID NO.: 2)) and/or analogs of PCK3145 (SEQ ID NO.: 5), the decapeptide (SEQ ID NO.: 3), the polypeptide 7-21 (SEQ ID NO. 4), the polypeptide 76-94 (SEQ ID NO. 6)) include, for example, the following:

a polypeptide analog of at least five contiguous amino acids of SEQ ID NO: 2, of SEQ ID NO: 3, of SEQ ID NO: 4, of SEQ ID NO: 5, or of SEQ ID NO: 6;

a polypeptide analog of at least two contiguous amino acids of SEQ ID NO: 2, of SEQ ID NO: 3, of SEQ ID NO: 4, of SEQ ID NO: 5, or of SEQ ID NO: 6;

a polypeptide analog consisting of the amino acid sequence X₁ W Q X₂ D X₁ C X₁ X₂ C X₂ C X₃ X₁ X₂, wherein X₁ is either glutamic acid (Glu), asparagine (Asn) or aspaltic acid (Asp), X₂ is either threonine (Thr) or serine (Ser), and X₃ is either tyrosine (Tyr) or phenylalanine (Phe);

a polypeptide analog comprising SEQ ID NO: 5 and having an addition of at least one amino acid to its amino-terminus;

a polypeptide analog comprising SEQ ID NO: 5 and having an addition of at least one amino acid to its carboxy-terminus;

a polypeptide analog comprising two to ten units of SEQ ID NO: 5;

a polypeptide analog comprising two to fifty units of SEQ ID NO: 5;

a polypeptide analog consisting of a sequence of from two to fourteen amino acid units wherein the amino acid units are selected from the group of amino acid units of SEQ ID NO: 5 consisting of glutamic acid (Glu), tryptophan (Trp), glutamine (Gln), threonine (Thr), aspartic acid (Asp), asparagine (Asn), cysteine (Cys), or tyrosine (Tyr);

a polypeptide analog having at least 90% of its amino acid sequence identical to the amino acid sequence set forth in SEQ ID NO: 5;

a polypeptide analog having at least 70% of its amino acid sequence identical to the amino acid sequence set forth in SEQ ID NO: 5;

and a polypeptide analog having at least 50% of its amino acid sequence identical to the amino acid sequence set forth in SEQ ID NO: 5;

or any polypeptide analog of PSP-94 (nPSP-94 (SEQ ID NO.: 1) or rHuPSP94 (SEQ ID NO.: 2)) as well as the derivative described herein (PCK3145 (SEQ ID NO.: 5), decapeptide (SEQ ID NO.: 3), polypeptide 7-21 (SEQ ID NO. 4), polypeptide 76-94 (SEQ ID NO. 6)) having the biological activity described herein (effect on hypercalcemia and bone metastasis).

Amino acids sequence insertions (e.g., additions) include amino and/or carboxyl-terminal fusions ranging in length from one residues to polypeptides containing a hundred or more residues, as well as intrasequence insertions of single or multiple amino acid residues. Other insertional variants include the fusion of the N- or C-terminus of the protein to a homologous or heterologous polypeptide forming a chimera. Chimeric polypeptides (i.e., chimeras, polypeptide analog) comprise sequence of the polypeptides of the present invention fused to homologous or heterologous sequence. Said homologous or heterologous sequence encompass those which, when formed into a chimera with the polypeptides of the present invention retain one or more biological or immunological properties.

A protein at least 50% identical, as determined by methods known to those skilled in the art (for example, the methods described by Smith, T. F. and Waterman M. S. (1981) Ad. Appl.Math., 2:482-489, or Needleman, S. B. and Wunsch, C. D. (1970) J.Mol.Biol., 48: 443-453), to those polypeptides of the present invention are included in the invention, as are proteins at least 70% or 80% and more preferably at least 90% identical to the protein of the present invention. This will generally be over a region of at least 5, preferably at least 20 contiguous amino acids.

It is to be understood herein, that if a “range” or “group” of substances (e.g. amino acids), substituents” or the like is mentioned or if other types of a particular characteristic (e.g. temperature, pressure, chemical structure, time, etc.) is mentioned, the present invention relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein. Thus, for example,

with respect to a temperature greater than 100° C., this is to be understood as specifically incorporating herein each and every individual temperature state, as well as sub-range, above 100° C., such as for example 101° C., 105° C. and up, 110° C. and up, 115° C. and up, 110 to 135° C., 115° C. to 135° C., 102° C. to 150° C., up to 210° C., etc.;

with respect to reaction time, a time of 1 minute or more is to be understood as specifically incorporating herein each and every individual time, as well as sub-range, above 1 minute, such as for example 1 minute, 3 to 15 minutes, 1 minute to 20 hours, 1 to 3 hours, 16 hours, 3 hours to 20 hours etc.;

with respect to polypeptides, a polypeptide analog consisting of at least two contiguous amino acids of a particular sequence is to be understood as specifically incorporating each and every individual possibility, such as for example, a polypeptide analog consisting of amino acid 1 and 2, a polypeptide analog consisting of amino acids 2 and 3, a polypeptide analog consisting of amino acids 3 and 4, a polypeptide analog consisting of amino acids 6 and 7, a polypeptide analog consisting of amino acids 9 and 10, a polypeptide analog consisting of amino acids 36 and 37, a polypeptide analog consisting of amino acids 93 and 94, etc.

and similarly with respect to other parameters such as, concentrations, elements, etc.

It is also to be understood herein that “g” or “gm” is a reference to the gram weight unit; that “C” is a reference to the Celsius temperature unit.

### BRIEF DESCRIPTION OF THE DRAWINGS

In drawings which illustrate examplary embodiments of the present invention:

FIG. 1 illustrates the effect of PSP-94 on Mat Ly Lu-PTHrP cell growth. Each point represents the mean of 3 different experiments. Significant differences from control cells (MatLyLu-CMV) and PTHrP transfected cells (MatLyLu-PTHrP) in the abscence of PSP-94 are represented by asterisks (p<0.05).

FIG. 2A illustrates the effect of PSP-94 on Mat Ly Lu-PTHrP tumor volume (in cm³). Results represent the mean±SEM of 5 animals in each group in 3 different experiments. Significant difference from control tumor-bearing animals receiving vehicle alone (CTL) are represented by asterisks (p<0.05).

FIG. 2B also illustrates the effect of PSP-94 on Mat Ly Lu -PTHrP tumor volume (in cm³). Results represent±SEM of six animals in each group. Significant difference in tumor volume is shown by asterisks (p<0.05).

FIG. 3 illustrates the effect of PSP-94 on animal weight. Results represent the mean±SEM of 5 animals in each group in 3 different experiments.

FIG. 4 illustrates the effect of PSP-94 on Mat Ly Lu-PTHrP tumor weight. Results represent the mean±SEM of 5 animals in each group in 3 different experiments. Significant difference from control tumor-bearing animals receiving vehicle alone (CTL) are represented by asterisks (p<0.05).

FIG. 5A illustrates the effect of PSP-94 on spinal metastases resulting in the development of hind limb paralysis. Results represent the mean±SEM of 5 animals in each group in 3 different experiments. Significant difference in % of non-paralyzed animals from control tumor-bearing animals receiving vehicle alone (CTL) are represented by asterisks (p<0.05).

FIG. 5B also illustrates the effect of PSP-94 on spinal metastases resulting in the development of hind limb paralysis.

FIG. 6A illustrates the effect of PSP-94 on plasma PTHrP in tumor bearing animals. Results represent the mean±SEM of 5 animals in each group in 3 different experiments. Significant difference from control tumor-bearing animals receiving vehicle alone (CTL) are represented by asterisks (p<0.05). Results obtained for non-tumor bearing animals (N) are also illustrated.

FIG. 6B illustrates the effect of PSP-94 on plasma calcium in tumor bearing animals. Results represent the mean±SEM of 5 animals in each group in 3 different experiments. Significant difference from control tumor-bearing animals receiving vehicle alone (CTL) are represented by asterisks (p<0.05). Results obtained for non-tumor bearing animals (N) are also illustrated.

FIG. 7A also illustrates the effect of PSP-94 on plasma PTHrP in tumor bearing animals. Results represent±SEM of 6 different animals in each group. Significant difference from control (CTL) is marked by asterisks (p<0.05). Results obtained for non-tumor bearing animals (N) are also illustrated

FIG. 7B also illustrates the effect of PSP-94 on plasma calcium of tumor bearing animals. Results represent±SEM of 6 different animals in each group. Significant difference from control (CTL) is marked by asterisks (p<0.05). Results obtained for non-tumor bearing animals (N) are also illustrated.

FIG. 8 illustrates the effect of PSP-94 on PTHrP production by Mat Ly Lu-PTHrP tumors. A representative photomicrograph of three such experiments is shown at a magnification 200×.

FIG. 9A illustrates the effect of PSP-94 on DNA fragmentation of Mat Ly Lu-PTHrP cells in vitro. A representative photograph of three such experiments is shown.

FIG. 9B illustrates the effect of PSP-94 on DNA fragmentation of Mat Ly Lu-PTHrP cells in vivo. All animals were sacrificed at the end of the study and their primary tumors removed, paraffin embedded, sectioned and processed by TUNEL assay as described herein (upper panel) or counterstained with Hoescht reagent (lower panel). Three animals were present in each group and three sections were analyzed for each animal. At least ten random fields of observation were evaluated. A representative photomicrograph for three such experiments in each group is shown. Magnification 200×

FIG. 10A illustrates the effect of PCK-3145 on plasma PTHrP levels in tumor bearing animals using a radioimmunoassay. Results represent the mean±SEM of 5 animals in each group in 3 different experiments. Significant differences from control tumor-bearing animals receiving vehicle alone (CTL) are represented by asterisks (p<0.05). Results obtained for non-tumor bearing animals (N) are also illustrated.

FIG. 10B illustrates the effect of PCK-3145 on plasma calcium levels in tumor bearing animals. Results represent the mean±SEM of 5 animals in each group in 3 different experiments. Significant differences from control tumor bearing animals receiving vehicle alone (CTL) are represented by asterisks (p<0.05). Results obtained for non-tumor bearing animals (N) are also illustrated.

FIG. 11. Effect of PCK-3145 on experimental skeletal metastases resulting in the development of hind limb paralysis. Results represent the mean±SEM of 5 animals in each group in 3 different experiments. Significant differences in percentage of non-paralyzed animals from control tumor-bearing animals receiving vehicle alone (CTL) are represented by asterisks (p<0.05).

### DETAILED DESCRIPTION OF THE INVENTION

In the current study we have examined the effect of PSP-94 or PCK3145 on prostate cancer growth and metastasis to the skeleton. For these studies, MatLyLu rat prostate cancer cells were transfected with full-length cDNA encoding parathyroid hormone related protein (PTHrP). MatLyLu-PTHrP cells were inoculated subcutaneously (S.C.) into the right flank or via intracardiac route (I.C.) into the left ventricle of syngenic male Copenhagen rats. Intracardiac inoculation of MatLyLu cells routinely results in tumor metastasis to the lumbar vertebrae resulting in hind limb paralysis. Time of hind limb paralysis and tumor volume was measured and comparison was made between PSP-94- or PCK3145-treated animals and control animals receiving vehicle alone. At the end of the study, animals were sacrificed and serum Ca⁺² (calcium, Ca⁺⁺) and PTHrP levels in control and experimental animals were determined. Primary tumors and skeletal metastasis to lumbar vertebrae were also examined for PTHrP production by immunohistochemistry. At the end of this study, affected lumbar vertebra were removed for radiological and histological analysis. Evidence of tumor cell apoptosis was monitored by subjecting histological specimens to Hoechst staining and TUNEL assays.

PSP-94 (native) was generated as described in U.S. Pat. No.: 5,428,011. The PCK3145 polypeptide was generated as described, for example, in Canadian patent application No.: 2,359,650.

Animal Protocols. Inbred male Copenhagen rats weighing 200-250 g were obtained from Harlan Sprague-Dawley (Indianapolis, Ind.). Before inoculation, Mat Ly Lu-PTHrP tumor cells growing in serum-containing medium were washed with Hanks buffer, trypsinized, and collected by centrifugation at 1500 rpm for 5 min. (Achbarou, A. et al., Cancer Res., 54:2372-2377, 1994; Rabbani, S. A. et al., Int. J. Cancer, 80: 257-264, 1999; Rabbani, S. A. et al., Endocrinology, 136:5416-5422, 1995). Cell pellets (10×10³ cells) were resuspended in 100 μl saline and injected using 1 ml insulin syringes into the left ventricle of rats anaesthetised with ketamine/xylazine cocktail. Animals were divided into control groups which received vehicle alone (PBS: phosphate buffered saline) and experimental groups which were infused I.P. with different doses (0.1-10.0 μg/kg/day) of PSP-94 starting at the time of tumor cell inoculation (day 0) until the day of skeletal metastases development. The time after tumor cell inoculation which was required to develop hind limb paralysis (an index of spinal cord compression due to lumbar vertebrae metastases) was determined and percentage of starting number of animals developing hind-limb paralysis was plotted.

Alternatively, cell pellets (5×10⁵ cells) were resuspended in 100 μl saline and injected using 1 ml insulin syringes into the right flank of rats as described herein. From the time of tumor cell inoculation, experimental animals were treated with different doses (0.1, 1.0 or 10.0 μg/kg/day) of PSP-94 via S.C. injections for 15 consecutive days. Control animals received PBS alone as vehicle control. All animals were numbered, kept separately and monitored daily for the development of tumors. The tumor mass was measured in 2 dimensions by calipers and tumor volume was calculated according to the equation (l×w²)/2 (l=length, w=width) (Rabbani, S. A. et al., Int. J. Cancer, 80: 257-264, 1999; Rabbani, S .A. et al., Endocrinology, 136:5416-5422, 1995). All control and experimental animals were weighed every alternate day to determine any adverse effect of PSP-94. Both control and experimental animals were sacrificed at day 16 post tumor cell inoculation and their tumors were removed and weighed. Additionally, these tumors were used for histological analysis as described herein. Blood from all control and experimental animals was collected on day 16 for determination of plasma Ca²⁺ and PTHrP levels.

Results presented herein are usually expressed as the mean±SE (standard error) of at least triplicate determinations, and statistical comparisons are based on the Student's t test or analysis of variance. A probability value of <0.05 was considered to be significant (Glantz, S. A., Primer of biostatistics, McGraw-Hill, new-York, 1981).

### EXAMPLE 1

### Effect of PSP-94 on MatLyLu-PTHrP Cell Growth, Morphology and Invasion.

Cells and cell culture. The Dunning R3327 Mat Ly Lu cell line (available, for example, under American Type Culture Collection No.: JHU-5) was transfected with full length cDNA encoding rat PTHrP as previously described (Rabbani, S. A. et al., Int. J. Cancer, 80: 257-264, 1999). One of the three well characterized monoclonal cell lines Mat Ly Lu-PTHrP-8 was used throughout the course of these studies. Cells were maintained in vitro in RPMI 1640 supplemented with 2 mM L-glutamine (Life Technologies, Inc. Grand Island, N.Y.), 10% fetal bovine serum (FBS), 100 units/ml penicillin-streptomycin sulphate (Life Technologies, Inc.), and 250 nM dexamethasone and G418 (600 mg/ml) according to previously established methods of culture of these experimental cells (Rabbani, S. A. et al., Int. J. Cancer, 80: 257-264, 1999).

Cell morphology. Morphological analysis of control and experimental Mat Ly Lu-PTHrP cells treated with PSP-94 was carried out by plating 5×10⁴ cells/well in 6-well plates (Falcon Plastics, Oxnard, Calif.) in the presence of 10% FBS. Cells were examined daily for any change in their morphology and photographed (Rabbani, S. A. et al., Endocrinology, 136:5416-5422, 1995).

Invasion. Effect of PSP-94 on Mat Ly Lu-PTHrP tumor cell invasive capacity was examined by 2-compartment Boyden Chamber (Transwell, Costar, Cambridge, Mass.) and basement membrane Matrigel (Becton Dickinson Labware, Bedford Mass.) as previously described (Liu, D. F. et al., Prostate, 27:269-276, 1995).

Growth curve. For growth curves, Mat Ly Lu-PTHrP cells were plated in 6-well plates (Falcon Plastics, Oxnard, Calif.) at seeding densities of 5×10³ cells/well. Mat Ly Lu-PTHrP cells were grown in the presence of 0.1, 1.0&10.0 μg/ml of PSP-94 or vehicle alone for up to 3 days and the ability of PSP-94 to alter cell doubling time was evaluated daily. Medium was changed every two days. The number of cells was counted in a model Z Coulter counter (Coulter Electronics, Beds, UK). Comparison was also made with doubling time of wild type untransfected Mat Ly Lu cells.

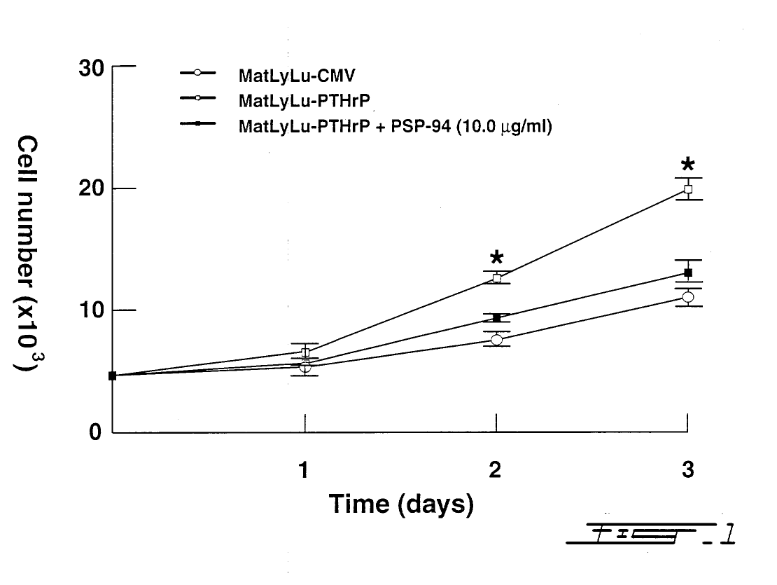

These results presented in FIG. 1 shows Mat Ly Lu cells transfected with vector alone (CMV) or vector expressing PTHrP were seeded at a density of 5×10³ cells/well in 6-well plates. Mat Ly Lu-PTHrP cells were treated with PSP-94 and were trypsinized and counted using a coulter counter as described herein. Change in cell number following treatment with 10.0 μg/ml of PSP-94 for 72 hrs is shown. Transfection of Mat Ly Lu with PTHrP cDNA resulted in reduced doubling time and increase in tumor cell growth due to the growth promoting effects of PTHrP. Thus, Mat Ly Lu-PTHrP cells had a higher rate of cell proliferation as compared to control Mat Ly Lu cells transfected with vector alone. A significant decrease in Mat Ly Lu-PTHrP cell growth was seen following treatment with 10.0 μg/ml of PSP-94 for 72 hrs (FIG. 1). Lower doses of PSP-94 (0.1 and 1.0 μg/ml) failed to exhibit any significant change on tumor cell growth (data not shown). Treatment of Mat Ly Lu-PTHrP cells with 10.0 μg/ml of PSP-94 for 3 days resulted in a noticeable change in tumor cell morphology where tumor cells were found to change their normal spindle-like shape to a more rounded and condensed appearance (data not shown). Using a Boyden Chamber Matrigel invasion assay, all doses of PSP-94 failed to alter the invasive capacity of Mat Ly Lu-PTHrP cells (data not shown).

### EXAMPLE 2

### Effect of PSP-94 on Mat Ly Lu-PTHrP Tumor Growth.

Male Copenhagen rats were inoculated with Mat Ly Lu-PTHrP cells via S.C. route of injection into the right flank as described above. Starting from the day of tumor cell inoculation animals were infused S.C., below the tumor cell inoculation site, with different doses of PSP-94 (0.1-10.0 μg/kg/day) for up to 15 days. Effect of PSP-94 on reducing tumor growth was evaluated by daily determination of tumor volume with comparison being made to control tumor-bearing animals receiving vehicle alone.

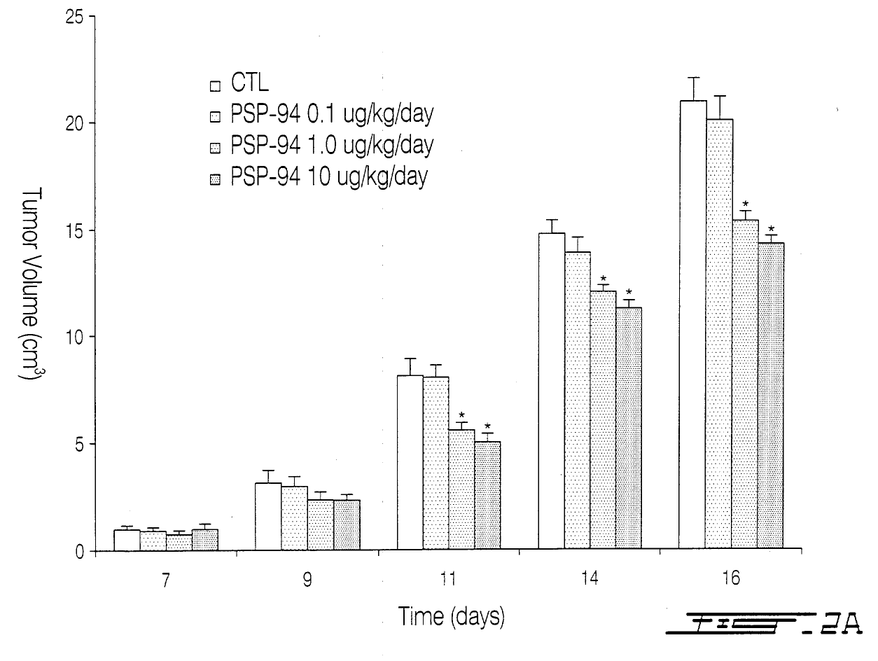

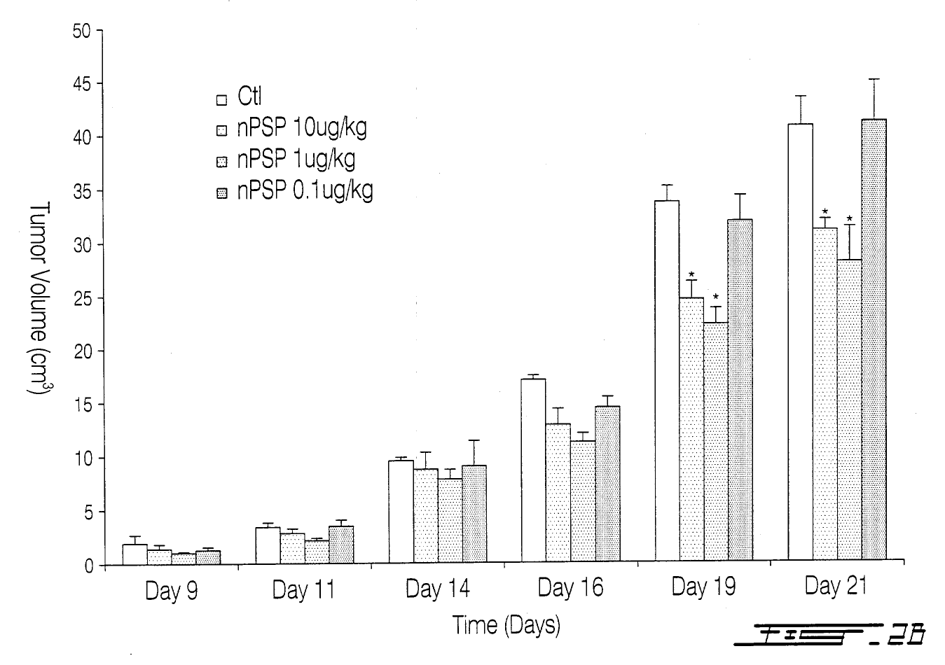

Results presented in FIG. 2A show Male Copenhagen rats injected S.C. into the right flank with 1×10⁶ Mat Ly Lu-PTHrP cells. Starting on the time of tumor cell inoculation animals were infused daily with different doses of PSP-94 for fifteen consecutive days as described herein. Tumor volume was measured at timed intervals and comparison was made with that of tumor-bearing animals receiving vehicle alone as control (CTL). Results of FIG. 2B also show Male Copenhagen rats inoculated s.c with 10⁶ Mat Ly Lu-PTHrP cells. After 3 days of tumor cell inoculation, animals were injected with vehicle alone (Ctl) or different doses (0.1, 1, 10 μg/kg) of PSP-94 (nPSP) at the site of tumor cell injection. Tumor volume (expressed in cubic centimeter (cm³)) was determined at timed intervals. Control animals showed a progressive increase in tumor volume throughout the duration of the study. In contrast to this, experimental animals receiving PSP-94 showed a marked dose-dependent reduction in tumor volume throughout the course of this study (FIGS. 2A, 2B).

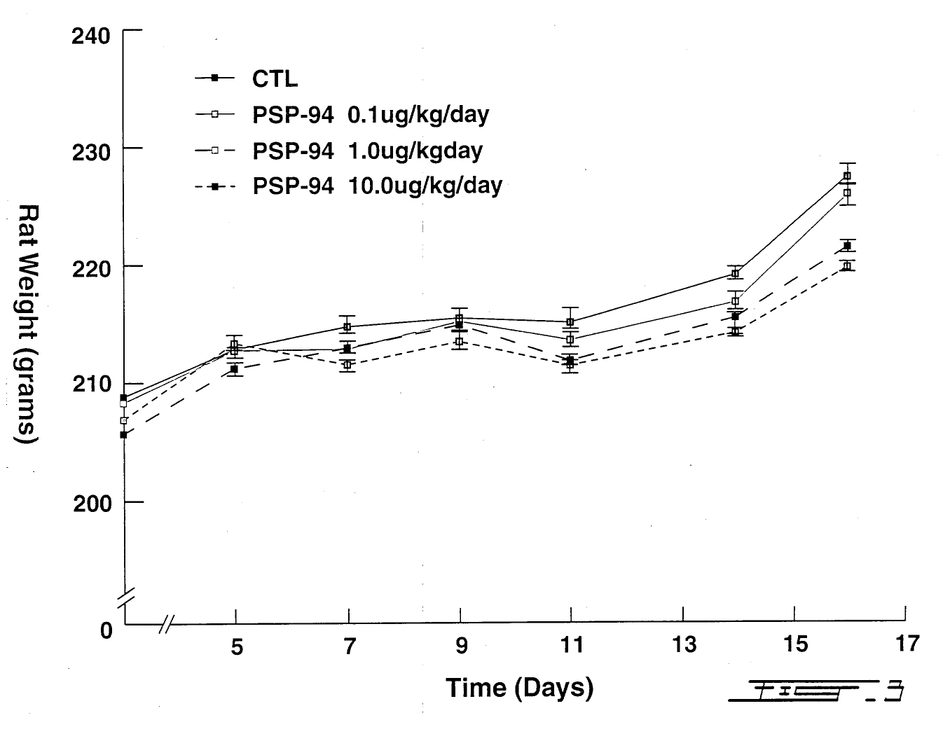

During this study, both control and experimental animals were monitored for any noticeable side effects and cachexia resulting in weight loss. Results presented in FIG. 3 shows Male Copenhagen rats injected S.C. into the right flank with 1×10⁶ Mat Ly Lu-PTHrP cells. Starting on the time of tumor cell inoculation animals were infused with different doses of PSP-94 for fifteen consecutive days as described herein. All animals were weighed at timed intervals and comparison was made with that of tumor bearing animals receiving vehicle alone as control (CTL). No significant change in the weight of control and experimental groups of animals that can be attributed to any potential side effect of PSP-94 treatment (FIG. 3) was observed.

### EXAMPLE 3

### Effect of PSP-94 on Mat Ly Lu-PTHrP Tumor Weight

In order to determine the effect of PSP-94 on tumor weight, animals inoculated with Mat Ly Lu-PTHrP via S.C. route of injection were sacrificed at the end of the study (day 16) and their tumors excised and weighed.

Results presented in FIG. 4 shows Male Copenhagen rats inoculated with 1×10⁶ Mat Ly Lu-PTHrP cells via subcutaneous injection into the right flank. Starting from the day of tumor cell inoculation animals were administered with different doses of PSP-94 for fifteen consecutive days as described herein. At the end of the study tumors from control (CTL), vehicle treated animals and PSP-94 treated animals were excised and weighed. Control animals receiving vehicle alone exhibited large tumors while treatment with different doses of PSP-94 (0.1-10.0 μg/kg/day) resulted in a significant dose-dependent decrease in tumor weight (FIG. 4).

Inoculation of male Copenhagen rats with Mat Ly Lu-PTHrP cells into the right flank via S.C. injections resulted in the development of primary tumors. Whereas control, vehicle treated animals developed large primary tumors, treatment with different doses of PSP-94 resulted in a dose-dependent decrease in their tumors mass. These anti-tumor effects were not associated with any noticeable side effects or weight loss of experimental animals.

### EXAMPLE 4

### Effect of PSP-94 and PCK3145 on the Development of Skeletal Metastases

Since the major cause of prostate cancer related mortality is the development of metastases, evaluation of the effect of PSP-94 on delaying the development of skeletal metastases was carried out by inoculating male Copenhagen rats with Mat Ly Lu-PTHrP cells via I.C. route into the left ventricle. Routine injection of Mat Ly Lu cells into the left ventricle results in the development of skeletal metastases causing compression of the spinal cord leading to hind-limb paralysis .

Mat Ly Lu-PTHrP cells were thus inoculated in male Copenhagen rats via I.C. injection into the left ventricle. Starting from the day of tumor cell inoculation (day 0), animals were administered with different doses of PSP-94 (0.1-10.0 μg/kg/day) via I.P. (intraperitoneal) route. The effect of PSP-94 on delaying the development of skeletal metastases was evaluated by daily monitoring of the animals for the development of hind-limb paralysis.

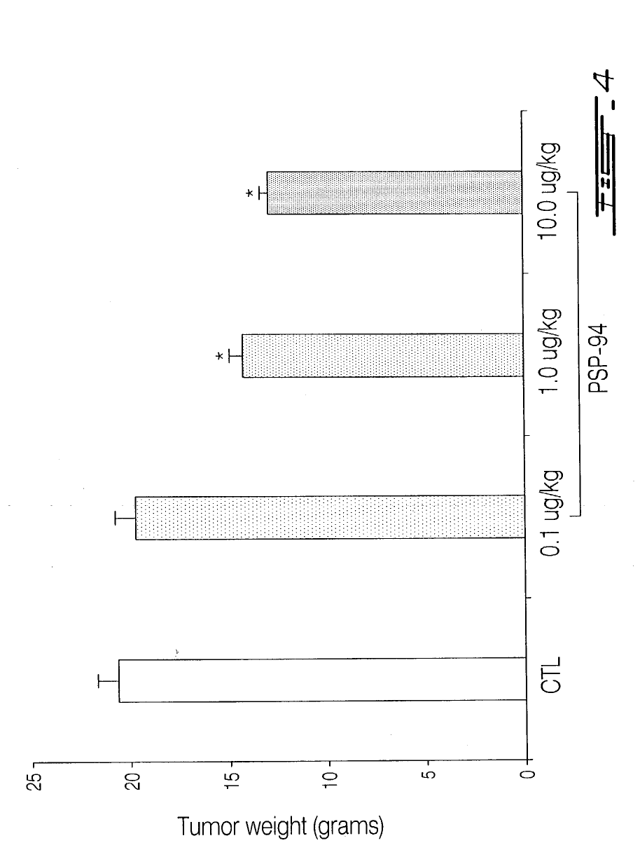

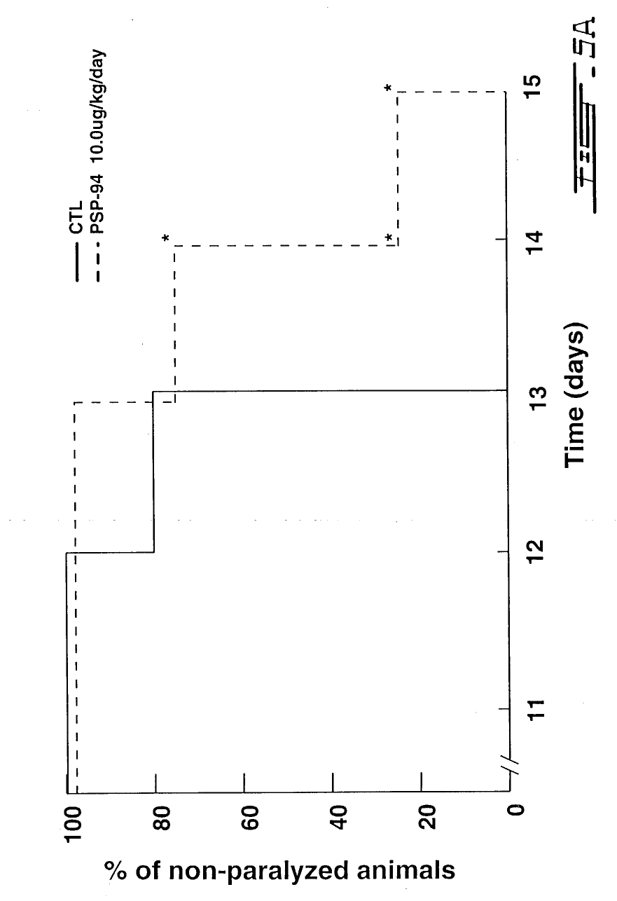

Results presented in FIG. 5A show Male Copenhagen rats inoculated via I.C. route into the left ventricle with 10×10³ Mat Ly Lu-PTHrP cells. Starting on the time of tumor cell inoculation (day 0) animals were infused with different doses of PSP-94 (0.1-10.0 μg/kg/day) until the day of development of hind-limb paralysis as described herein. Animals receiving vehicle alone as control (CTL) or PSP-94 were monitored daily for the development of hind-limb paralysis and % animals not paralyzed at different time points in each group was calculated. All (100%) control animals inoculated with Mat Ly Lu-PTHrP cells and receiving vehicle alone developed hind-limb paralysis by day 13. While 0.1 and 1.0 μg/kg/day PSP-94 had no significant effect on the time of hind limb paralysis (data not shown), treatment with 10.0 μg/kg/day PSP-94 resulted in a statistically significant delay in the number of animals developing hind limb paralysis. Percentage of total number of animals receiving PSP-94 not developing hind limb paralysis at different days is shown in FIGS. 5A and 5B.

A second set of experimentation was performed. Results presented in FIG. 5B also show Male Copenhagen rats inoculated via the intracardiac (i.c) route with 5×10⁴ Mat Ly Lu-PTHrP cells. After 3 days of tumor cell inoculation, animals were injected by intraperitoneal route with vehicle alone (Ctl) or different doses of PSP-94 (nPSP). Time to the development of hind limb paralysis in Ctl and animals receiving 10 μg/kg/day of PSP-94 is shown.

Percentage of total number of animals receiving PCK3145 not developing hind limb paralysis at different days is shown in FIG. 11. Results presented in FIG. 11 show Male Copenhagen rats inoculated via I.C. injection with 10×10³ Mat Ly Lu-PTHrP cells. Starting on the say of tumor cell inoculation (day 0), animals were infused with different doses of PCK-3145 (1.0-100.0 μg/kg/day) until the day of hind-limb paralysis development as discussed herein. Animals receiving vehicle alone as control (CTL) or PCK-3145 were monitored daily for the development of hind-limb paralysis and percentage of animals not paralyzed at different time points in each group was calculated.

While all control, vehicle treated animals developed hind-limb paralysis by day 13, administration of the highest dose of PSP-94 starting from the time of tumor cell inoculation resulted in modest delay in skeletal metastases. Such results suggest low bioavailability of PSP-94 to the skeleton, a common drawback associated with developing effective therapeutic agents for skeletal metastases (Rabbani, S. A. et al., Cancer res., 58:3461-3465, 1998).

### EXAMPLE 5

### Effect of PSP-94 and PCK3145 on Plasma PTHrP and Calcium Levels and Tumoral PTHrP Production.

In order to determine the effect of PSP-94 and PCK3145 on plasma PTHrP and calcium levels animals inoculated with Mat Ly Lu-PTHrP cells via S.C. route were sacrificed at the end of the study, plasma was collected and PTHrP levels were analyzed using a radioimmunoassay. Comparison was made between plasma collected from normal, non-tumor bearing animals, control tumor bearing animals receiving vehicle alone and plasma collected from experimental animals receiving different doses of PSP-94 (0.1-10.0 μg/kg/day). Plasma calcium levels were determined by atomic absorption spectrophotometry (model 703, Perkin-Elmer, Norwalk, Conn.). For plasma PTHrP, all samples were tested in two dilutions in PTHrP R.I.A. kit (Nichols Institute Diagnostics, San Juan Capïstrano, Calif.) according to manufacturers instructions.

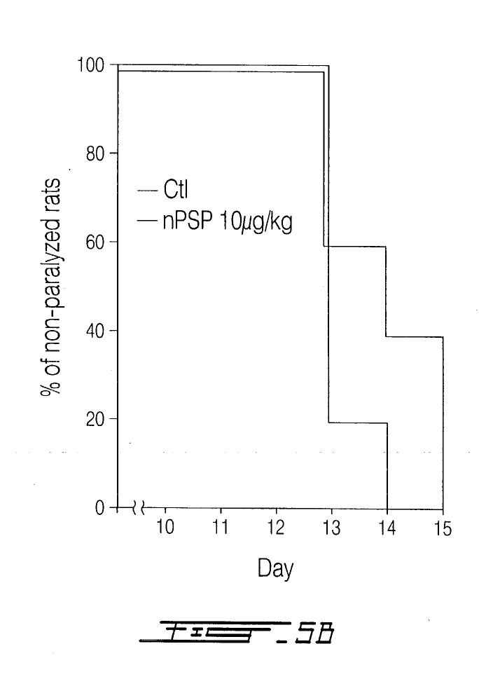

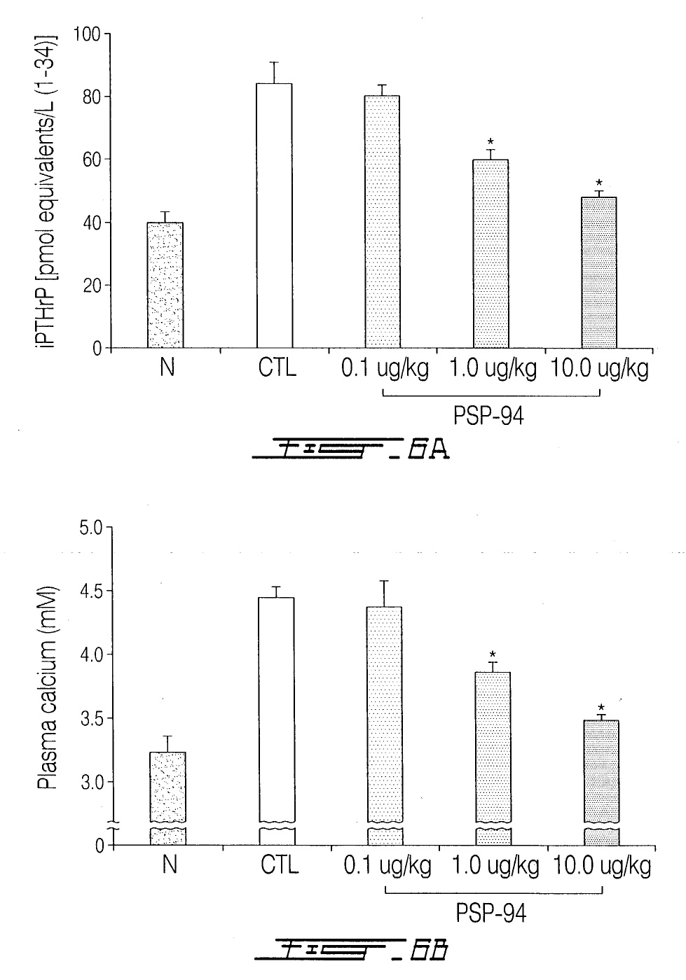

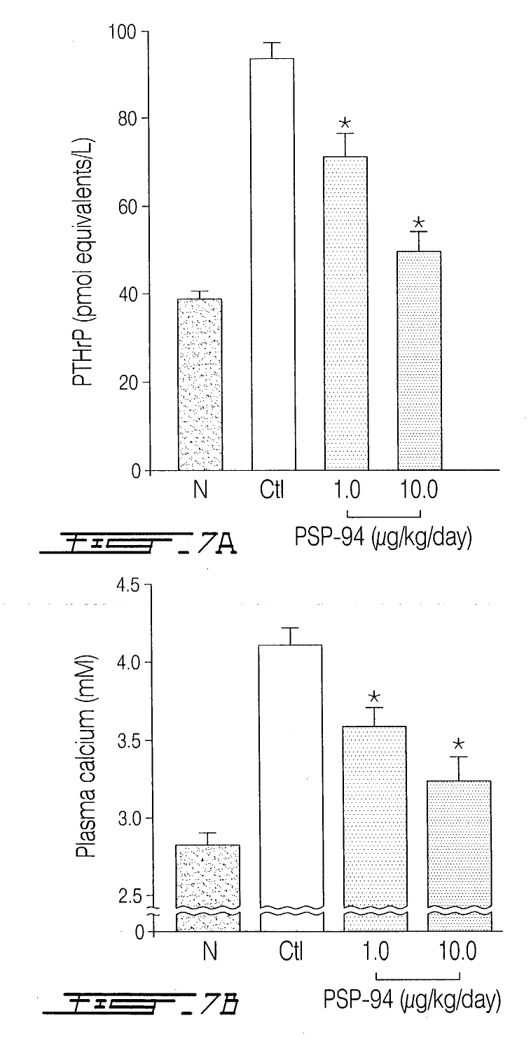

Normal non-tumor bearing animals showed basal levels of plasma PTHrP whereas animals inoculated with Mat Ly Lu-PTHrP cells and receiving vehicle alone showed marked elevated levels of immunoreactive plasma PTHrP levels. Treatment of tumor bearing animals with PSP-94 resulted in a dose-dependent decrease in plasma PTHrP levels (FIG. 6A). Analysis of plasma collected from normal non-tumor bearing animals and tumor bearing animals receiving vehicle alone revealed a marked increase in plasma calcium of control tumor bearing animals at the time of sacrifice on day 16 past tumor cell inoculation. In contrast, experimental groups of animals receiving different doses of PSP-94 resulted in significant reduction in their plasma calcium levels. The highest dose of PSP-94 (10.0 μg/kg/day) resulted in near normalization of plasma calcium of these experimental group of animals (FIG. 6B). See also results presented in FIGS. 7A and 7B.

Results of FIG. 6A and 6B show Male Copenhagen rats inoculated S.C. with 1×10⁶ Mat Ly Lu-PTHrP cells. Starting on the time of tumor cell inoculation animals were administered with different doses of PSP-94 for fifteen consecutive days as described herein. All animals were sacrificed at the end of the study (day 16) and plasma was collected from control (CTL) vehicle treated animals and PSP-94 treated animals and analyzed for immunoreactive plasma PTHrP (iPTHrP) levels using radioimmunoassay as described herein (FIG. 6A) or for plasma calcium levels as described herein (FIG. 6B). Plasma PTHrP levels in normal non-tumor bearing animals (N) (FIG. 6A) and plasma calcium from normal, non-tumor bearing animals are also shown (N) (FIG. 6B).

A second set of experiment was performed. Similar results are presented in FIGS. 7A and 7B. Results of FIG. 7A shows Male Copenhagen rats inoculated s.c with 10⁶ Mat Ly Lu-PTHrP cells. Following 3 days of tumor cell inoculation, animals were treated with vehicle alone (Ctl) or different doses (1.0, or 10.0 μg/kg/day) of PSP-94 for 18 days. Animals were sacrificed on day 21 and plasma PTHrP was determined. PTHrP levels (expressed in picomole equivalents/liter) of non-tumor bearing animals is also shown (N). Results of FIG. 7B shows Male Copenhagen rats inoculated s.c with 10⁶ Mat Ly Lu-PTHrP cells. Following 3 days of tumor cell inoculation, animals were treated with vehicle alone (Ctl) or different doses (1.0, or 10.0 μg/kg/day) of PSP-94 for 18 days. Animals were sacrificed on day 21 and plasma calcium (expressed in millimolar (mM)) was determined. Plasma calcium of non-tumor bearing animals is also shown (N).

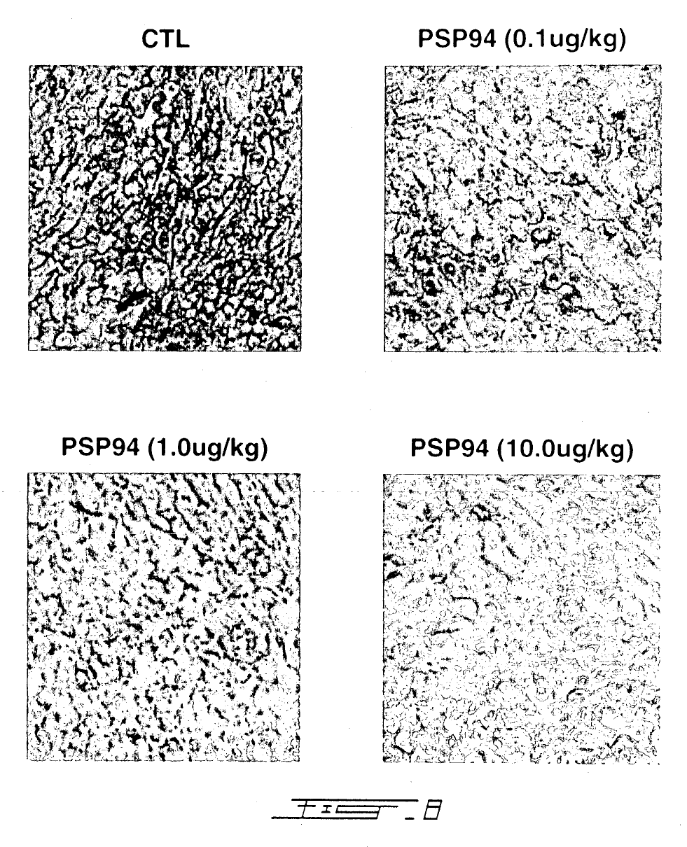

All animals were sacrificed at the end of the study. Tumors from control group treated with vehicle alone and experimental groups treated with different doses of PSP-94 (0.1-10.0 μg/kg/day) were excised, paraffin embedded, and sectioned and analyzed for tumoral PTHrP production by immunohistochemical reaction specific for PTHrP using method described herein. Results of FIG. 8 show histologic sections of tumors from animals receiving vehicle alone (CTL) or different doses of PSP-94 and stained with an antibody specific for PTHrP as described herein. Three animals were present in each group and three tumor sections were analyzed for each animal by evaluating at least ten random fields of observation.

Histologic Analysis. For immunohistological analysis, primary tumor samples were dewaxed by heating at 60° C. and rehydrated in a graded alcohol series (100%-70%). Anti-rat antibody against PTHrP was used as the primary antibody. Tumor sections were incubated overnight at 4° C. followed by further incubation with biotinylated universal antibody (Vector Laboratories, Burlingame, Calif.) for 45-60 minutes. Sections were rinsed with TBST (Tris buffered saline-Tween) followed by incubation with Vectastain ABC-AP Reagent (Vector Laboratories, Burlingame, Calif.) for 30 minutes. These sections were again washed with TBST and incubated with a Napthol AS-Mix Phosphate/Fast Red solution (Sigma-Aldriche, Oakville, ON). The sections were finally counterstained with Methyl Green (Vector Laboratories, Burlingame, Calif.) and mounted.

Intense color staining of tumors from control groups of animals receiving vehicle alone was observed. In contrast a dose dependent decrease in PTHrP immunostaining was observed in experimental tumors from animals receiving different doses of PSP-94 (FIG. 8).

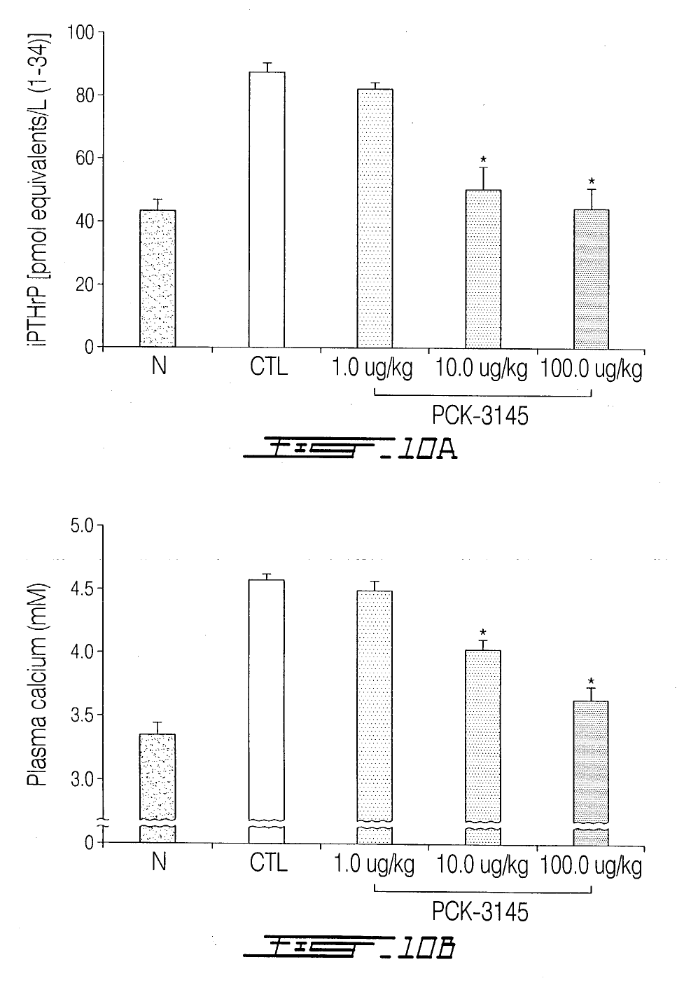

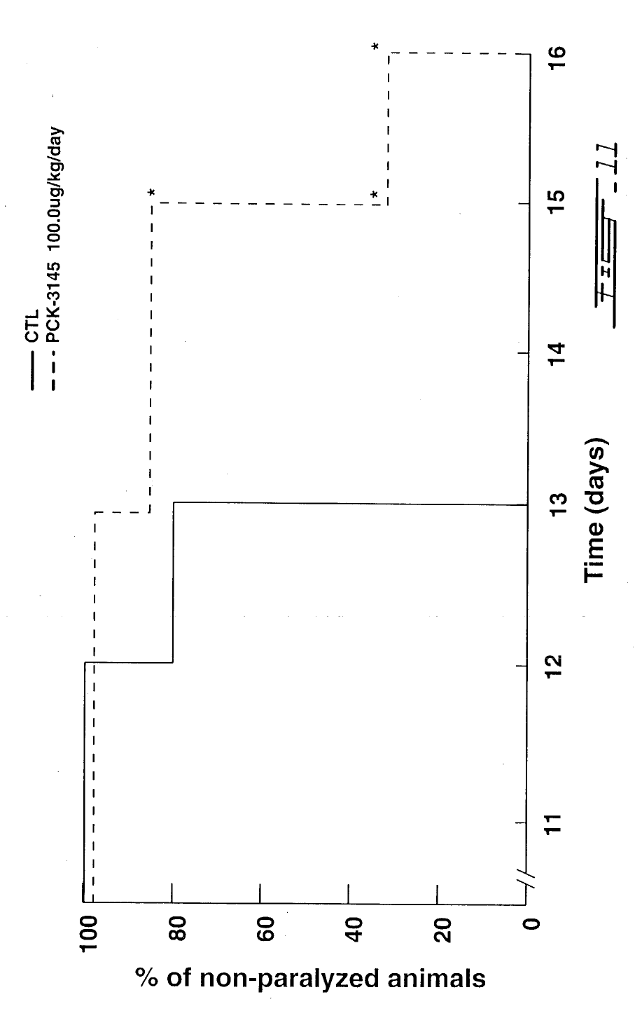

Similar results obtained in tumor bearing animals inoculated with PCK3145 are illustrated in FIGS. 10A and B.

Results presented in FIG. 10A show Male Copenhagen rats inoculated S.C. with 1×10⁶ Mat Ly Lu-PTHrP cells. Starting on the time of tumor cell inoculation, animals were administered with different doses of PCK-3145 for fifteen consecutive days as described herein. All animals were sacrificed at the end of the study (day 16) and plasma was collected from control (CTL), vehicle treated animals and PSP-94 treated animals and analyzed for immunoreactive PTHrP (iPTHrP) levels using a radioimmunoassay as described herein. Plasma PTHrP levels in normal non-tumor bearing animals is also shown (N).

Results presented in FIG. 10B show Male Copenhagen rats inoculated S.C. with 1×10⁶ Mat Ly Lu-PTHrP cells. Starting on the time of tumor cell inoculation, animals were administered with different doses of PCK-3145 for fifteen consecutive days as described herein. All animals were sacrificed at the end of the study (day 16) and plasma was collected from vehicle treated control animals (CTL) and PCK-3145 treated animals and analyzed for plasma calcium levels as described herein. Plasma calcium from non-tumor bearing animals is also shown (N).

As discussed above, upon sacrifice of animals, plasma was collected and analyzed for PTHrP and calcium levels. Normal, non-tumor bearing animals have undetectable levels of plasma PTHrP whereas inoculation of animals with Mat Ly Lu-PTHrP cells resulted in marked increase in their plasma PTHrP levels. In contrast to this, treatment with the different doses of PSP-94 resulted in a dose-dependent decrease in plasma PTHrP levels. In addition, the same dose-dependent decrease was observed in tumoral PTHrP production when tumor samples from control, vehicle treated and PSP-94 treated animals were subjected to immunohistochemical analysis.

Being the major pathogenetic factor of hypercalcemia of malignancy, plasma calcium levels correlate with that of plasma PTHrP levels (Iwamura, M., et al., Urology 43:675-679, 1994; Iwamura, M., et al., Hum. Pathol. 26:797-801, 1995; Suva, L. J., et al., Science, 237:893-896, 1987). Inoculation of Mat Ly Lu-PTHrP cells into the animals resulted in a marked increase in their plasma calcium levels as compared to serum from normal, non-tumor bearing animals. Administration of different doses of PSP-94 resulted in a dose-dependent decrease in plasma calcium levels with the highest dose of PSP-94 leading to a near normalization of plasma calcium levels.

Using this model we were not only able to demonstrate the anti-tumor effects of PSP-94 by reduction in tumor volume and weight but also biochemical parameters like plasma calcium and PTHrP levels also showed a marked decrease following therapy. A significant finding in these studies was that while decrease in tumor volume was dose-dependent, 10.0 μg/kg/day PSP-94 did not show a marked decrease in tumor volume as compared to 1.0 μg/kg/day PSP-94. In contrast, the ability of PSP-94 to reduce plasma calcium, plasma PTHrP and tumoral PTHrP continued to show a dose-dependent effect with 10.0 μg/kg/day PSP-94 causing near normalization of plasma calcium and PTHrP levels. These findings allow us to speculate that PSP-94 may also have additional effects including its ability to regulate PTHrP production by tumor cells or alter calcium homeostasis. Indeed PSP-94 has been shown to suppress follicle stimulating hormone (FSH) which is known to regulate intracellular calcium (Touyz, R. M. et al., Biol. Reprod. 62:1067-1074, 2000). Suppression of FSH by PSP-94 may serve as an additional mechanism to cause anti-tumor effects due to the growth-promoting effects of FSH in prostate cancer (Porter, A. T. et al., Urol. Oncol., 6:131-138, 2001). Furthermore, although cloning and characterization of a putative PSP-94 receptor has not been performed several studies have provided evidence for the existence of PSP-94 binding proteins on prostate cancer cells which could allow PSP-94 binding to initiate a signaling cascade that results in the observed anti-tumor effects observed (Yang, J. P. et al., J.Urol. 160:2240-2244, 1998; Yang, J. P. et al., Prostate, 35:11-17, 1998).

### EXAMPLE 6

### Effect of PSP-94 Mat Ly Lu-PTHrP Tumor Cell Apoptosis in Vitro and in Vivo.

In TUNEL assay, tissue sections were dewaxed and rehydrated by heating at 60° C. followed by washing in xylene and rehydration through a graded series of ethanol and water. Tissues were incubated with proteinase K for 30 min at 37° C. and fixed, blocked and permeabilized. Apoptotic cells were detected by TUNEL assay in situ cell death detection kit (Roche Molecular Biochemicals, Laval, QC) according to the manufacturers instruction. Positive TUNEL staining was visualised by fluorescence microscopy.

In other experiments following TUNEL assay, tissue sections were counterstained with Hoechst 33258 (Sigma-Aldrich, Oakville, Canada). Hoechst staining was added to tissues at a final concentration of 24 μg/ml in PBS and incubated for 15 minutes at room temperature. Tissue sections were washed and visualized by fluorescence microscopy using a blue screen (Rabbani, S. A., et al., Int. J.Cancer, 87:276-282, 2000). All results of immunohistochemistry and TUNEL assay were evaluated and interpreted by two independent examiners.

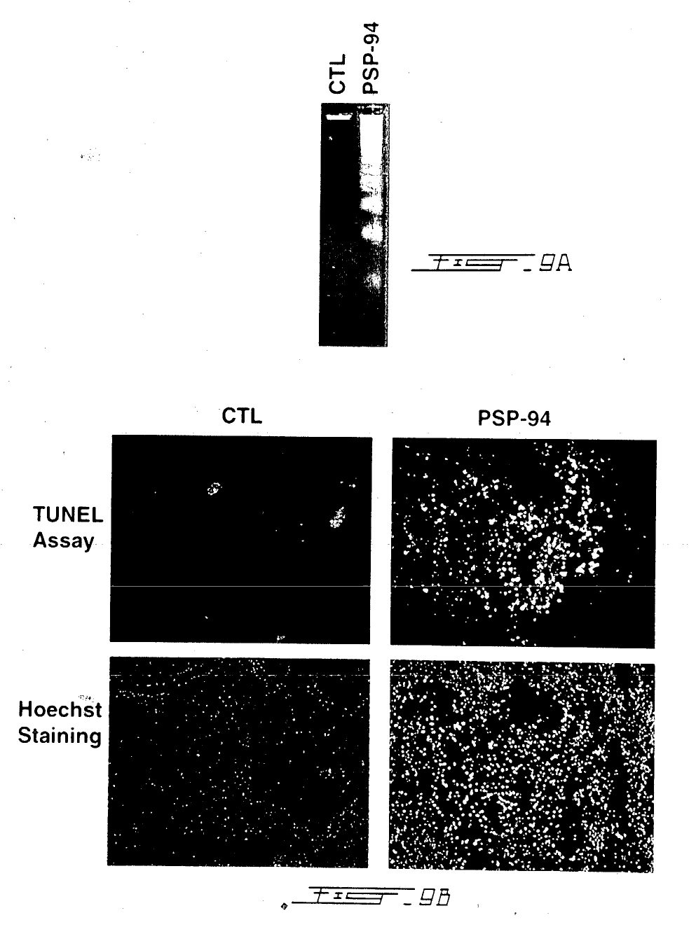

In order to investigate the underlying molecular mechanism of action of PSP-94 in reducing tumor growth Mat Ly Lu-PTHrP cells were cultured in the presence of PSP-94 (10.0 μg/ml) or vehicle alone for different time intervals. Genomic DNA was collected from cells cultured in the presence of vehicle alone or PSP-94. Briefly, for DNA fragmentation, Mat Ly Lu-PTHrP cells were plated in 6 well plates (Falcon Plastics, Oxnard, Calif.). Cells were treated with PSP-94 (10.0 μg/ml) for up to 72 hours. DNA from treated cells incubated with PSP-94 and cells treated with vehicle alone was collected using a Phenol:Choloroform:Isoamyl alcohol solution (50:48:2). Equal amounts of DNA were subjected to gel electrophoresis on a 2% agarose gel. DNA fragmentation was visualised by UV light using a transilluminator. More particularly, results presented in FIG. 9A show Mat Ly Lu-PTHrP cells cultured in the presence of vehicle alone or PSP-94 (10.0 μg/ml) for up to 96 hours. DNA was isolated and run in an agarose gel, as described above. These results show that control Mat Ly Lu-PTHrP cells cultured with vehicle alone exhibited no signs of DNA fragmentation. However, experimental Mat Ly Lu-PTHrP cells cultured in the presence of PSP-94 (10.0 μg/ml) exhibited marked DNA fragmentation after 72 hours of treatment (FIG. 9A).

The degree of DNA fragmentation was also analyzed in vivo using TUNEL assay which can serve as a marker for apoptosis. More particularly, results presented in FIG. 9B are derived from tissue collected from Male Copenhagen rats inoculated with 1×10⁶ MatLyLu-PTHrP cells and infused with different doses of PSP-94 for fifteen consecutive days as described herein. All animals were sacrificed at the end of the study and their primary tumors removed, paraffin embedded, sectioned and processed by TUNEL assay (upper panel) as described herein. Following TUNEL assay, they were counterstained with Hoescht reagent (lower panel). Tumor sections treated with PSP-94 (10.0 μg/kg/day) were significantly more TUNEL positive as compared to vehicle treated control tumors (FIG. 9B). Counterstaining with Hoechst reagent revealed the presence of apoptotic bodies in tissue sections from animals treated with PSP-94. Furthermore, condensed chromatin, which is characteristic of apoptotic cells, was observed in PSP-94 treated tumors. Control, vehicle treated tumors exhibited normal DNA staining patterns (FIG. 9B). These in vitro and in vivo findings demonstrate that indeed reduction in tumor volume following treatment with PSP-94 is due to its ability to promote tumor cell apoptosis.

As indicated above, TUNEL analysis carried out on tumoral sections from control and experimental animals revealed that PSP-94 treated tumors are more TUNEL positive as compared to control tumors, indicating a higher degree of apoptosis in PSP-94 treated animals. In addition to this, counterstaining with Hoechst reagent revealed condensed, apoptotic chromatin in PSP-94 treated tumors whereas control tumors exhibited normal DNA staining.

Collectively, the results of this study demonstrate PSP-94 and PCK3145 to be effective inhibitors of hormone-independent, late stage prostate cancer growth and its associated hypercalcemia of malignancy without manifesting any noticeable cytotoxic effects. The use of polypeptides, their peptidomimetic analogues alone or in combination with currently available chemotherapeutic agents will provide unique opportunities to block prostate cancer progression with highly effective non-toxic biotherapeutic agents which can be delivered over a long period of time without any drug associated side effects. These approaches will go a long way in reducing prostate cancer associated morbidity and mortality.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

We claim:

1. The use of PSP-94 for treating a patient suffering from hypercalcemia of malignancy.

2. The use as defined in claim 1, wherein said hypercalcemia of malignancy arise from cancer selected from the group consisting of prostate cancer, breast cancer and carcinomas.

3. The use of PSP-94 for reducing PTHrP levels in a patient in need thereof.

4. The use of PSP-94 to reduce the development of skeletal metastasis.

5. The use of PSP-94 in the manufacture of a pharmaceutical composition for the treatment of hypercalcemia of malignancy.

6. The use of PSP-94 in the manufacture of a pharmaceutical composition for the treatment of skeletal metastasis.

7. A method for evaluating, the efficacy of PSP-94 tumor treatment in a patient having a tumor, said method comprising measuring plasma calcium levels in said patient.

8. A method for evaluating, in a patient, the efficacy of PSP-94 treatment of hypercalcemia of malignancy, said method comprising measuring plasma calcium levels in said patient.

9. A method for evaluating, in a patient, the efficacy of PSP-94 treatment, said method comprising;
a) measuring plasma calcium from a patient with a tumor or with hypercalcemia of malignancy before the patient's treatment with PSP-94
b) measuring plasma calcium from a patient with a tumor or with hypercalcemia of malignancy after the patient's treatment with PSP-94; and
c) comparing values obtained in step a) with values obtained in step b).

10. A method for evaluating, the efficacy of PSP-94 tumor treatment in a patient having a tumor, said method comprising measuring plasma PTHrP levels in said patient.

11. A method for evaluating, in a patient, the efficacy of PSP-94 treatment of hypercalcemia of malignancy, said method comprising measuring plasma PTHrP levels in said patient.

12. A method for evaluating, in a patient, the efficacy of PSP-94 treatment, said method comprising;
a) measuring plasma PTHrP from a patient with a tumor or with hypercalcemia of malignancy before the patient's treatment with PSP-94
b) measuring plasma PTHrP from a patient with a tumor or with hypercalcemia of malignancy after the patient's treatment with PSP-94; and
c) comparing values obtained in step a) with values obtained in step b).
